(19) United States
(12) Patent Application Publication
(10) Pub. No.: US 2005/0175562 A1
(43) Pub. Date: Aug. 11, 2005

(54) Skin makeup composition

(76) Inventors: Anke Hadasch, Paris (FR); Marie-Laure Delacour, Paris (FR); Isabelle Mori, Ville D'Avray (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/028,563

(22) Filed: Jan. 5, 2005

Related U.S. Application Data
(60) Provisional application No. 60/536,245, filed on Jan. 14, 2004.

(30) Foreign Application Priority Data
Jan. 5, 2004 (FR) 04 50008

(51) Int. Cl. A61K 7/021; A61K 7/06; A61K 7/11

(52) U.S. Cl. 424/63; 424/70.12

(57) ABSTRACT

The present disclosure relates to a composition comprising elastomeric crosslinked organopolysiloxane particles having a refractive index ranging from 1.2 to 1.45, a liquid binder having a refractive index ranging from 1.2 to 1.6, and a colorant chosen from reflective particles other than those comprising mica substrates, goniochromatic coloring agents, photochromic coloring agents, fluorescent agents, and mixtures thereof. The present disclosure further relates to a method of making up the skin and lips.

This application claims benefit of U.S. Provisional Application No. 60/536,245, filed Jan. 14, 2004.

The present disclosure relates to a makeup composition comprising solid particles and particular colorants. The disclosure also relates to a method of making up a human being's skin or lips that comprises applying the composition to the skin or lips.

The makeup composition may be a skin makeup product, such as a complexion product (e.g., foundation), eye shadow, blusher or eyeliner, a concealer product, a body makeup product or a lip makeup product. For example, the composition may be a skin makeup product, such as a foundation, eye shadow or blusher. In certain embodiments, the present disclosure relates to a complexion makeup composition, e.g., a foundation, and to an eye shadow composition.

Makeup products such as eye shadows generally comprise a pulverulent phase comprising, for example, fillers and pigments. Certain fillers, however, such as talc, owing to their opacity to light, have a tendency to reduce the saturation of the color of the pigments present in the makeup product, with the consequence that the resulting makeup presents an apparent color, which is less vivid than the color of the colorants alone. The color attenuation of the makeup is readily perceptible, for instance, for eye shadows in powder form (free powders or compact powders) with a high filler content, of at least 50% by weight, for example.

Other known colorants are, for example, reflective particles, such as metal-coated glass particles, as described in European Patent No. EP-A-1082952, or else of goniochromatic pigments with a multi-layer interference structure or with a liquid-crystal structure, as described in European Patent No. EP-A-953330. These colorants produce colored optical effects, which are original, intense, and different from those obtained with conventional pigments such as iron oxides or titanium dioxide. If these original colorants are formulated with opaque fillers, though, such as talc, e.g., when they are present in a large amount, these opaque fillers will attenuate the original colored effect of the makeup product and the resulting makeup then presents a reduced colored effect. Thus, the intensity of the optical effects of the colorants is masked by the opacity of these fillers. It may be then necessary to introduce a large amount of colorants in order for the desired colored effects (i.e., optical effect) to be readily visible.

For makeup products in a form other than that of a powder, such as a cream, a gel or a cast product, for example, whether particularly anhydrous or aqueous, the pulverulent materials may only be introduced, in general, in a proportion of up to 30% by weight. Accordingly, when these compositions include opaque fillers such as talc, it may not be possible to introduce a large amount of optical-effect colorants such as those mentioned above, which therefore limits the possibility of obtaining makeup presenting an intense colored effect.

To address at least some of these issues, the present disclosure provides a makeup composition that comprises colorants and presents a colored effect which may be less attenuated relative to the colored effect of the colorants alone.

The present inventors have discovered that such a composition may be obtained by using specific colorants in combination with a liquid binder and with particular solid particles.

More specifically, in one embodiment, the present disclosure provides a makeup composition comprising solid particles of a material having a refractive index ranging from 1.2 to 1.45, a liquid binder having a refractive index ranging from 1.2 to 1.6, and a colorant chosen from reflective particles other than reflective particles comprising a mica substrate, goniochromatic coloring agents, photochromic coloring agents, fluorescent agents, and mixtures thereof.

The present disclosure likewise provides a cosmetic method of making up the skin or lips, which comprises applying to the skin or to the lips a composition as defined above. The method is, for example, intended for the skin.

The composition according to the present disclosure may allow makeup to be obtained which presents an intense colored effect which is reduced only to a small extent relative to the colored effect of the colorants alone. For example, the selective choice of the solid particles and of the liquid binder allows the colored effect (i.e., optical effect) of the specific colorants used to be “boosted”, thereby providing a bright, intense makeup.

The composition according to the present disclosure comprises solid particles of a material having a refractive index ranging from 1.2 to 1.45 (e.g., from 1.2 to 1.43). In another example, the refractive index of these particles ranges from 1.25 to 1.45 e.g., from 1.25 to 1.43), such as from 1.3 to 1.45 (e.g., from 1.3 to 1.43), for example, from 1.35 to 1.45 (e.g., from 1.35 to 1.43), such as from 1.38 to 1.45 (e.g., from 1.38 to 1.43), and further for example, from 1.40 to 1.45 (e.g., from 1.40 to 1.43).

As defined herein, “solid particles” are particles of material which is in the solid state (and hence not liquid) at ambient temperature (25° C.).

The refractive index of a solid particle is determined by making a mixture of the solid particles in an amount ranging from 1% to 10% by weight of the total weight of the mixture with a liquid medium comprising an oil or a solvent or a mixture of oils or solvents selected such that the mixture comprises particles+liquid medium and is transparent. In other words, this mixture, when subjected to radiation in the wavelength range 400 nm-700 nm using a spectrophotometer, presents a direct transmission of 100%.

The refractive index of the particle is then equal to the refractive index of the medium which allows a direct transmission of 100% to be obtained.

If the liquid medium comprises a single oil or a single solvent, then the refractive index of the solid particle is equal to the refractive index of the oil or solvent.

If the liquid medium comprises a mixture of oils or solvents, then the refractive index is calculated as a function of the proportion by volume of each oil or solvent in the liquid medium, weighted by its refractive index. For example, the liquid medium comprises a mixture of ethanol (which has a refractive index of 1.36) and phenyl alcohol (which has a refractive index of 1.529).

The refractive index is determined at ambient temperature (25° C.).

The solid particles of a material having a refractive index ranging from 1.2 to 1.45 may be chosen from particles of elastomeric crosslinked organopolysiloxane, particles of silica having a refractive index ranging from 1.2 to 1.45, and particles of polytetrafluoroethylene. In one embodiment, these particles are chosen from particles of elastomeric crosslinked organopolysiloxane.

As defined herein, an “elastomeric” organopolysiloxane is a flexible organopolysiloxane which is deformable, having viscoelastic properties, and possessing, for example, the consistency of a sponge or a flexible sphere. Its elasticity modulus is such that this material resists deformation and possesses a limited capacity for extension and for contraction. The “elastomeric” organopolysiloxane material is capable of recovering its original shape after having been drawn out.

The elastomeric crosslinked organopolysiloxane particles are chosen from those obtained by crosslinking addition reaction of a diorganopolysiloxane comprising at least one hydrogen bonded to silicon and a diorganopolysiloxane having ethylenically unsaturated groups bonded to silicon, for example, in the presence of a platinum catalyst; by dehydrogenation crosslinking condensation reaction between a diorganopolysiloxane having hydroxyl end groups and a diorganopolysiloxane comprising at least one hydrogen bonded to the silicon, for example in the presence of an organotin compound; by crosslinking condensation reaction of a diorganopolysiloxane having hydroxyl end groups and a hydrolysable organopolysilane;by thermal crosslinking of organopolysiloxane, such as in the presence of an organic peroxide catalyst; and by high-energy radiation crosslinking of an organopolysiloxane, such as by a device emitting gamma rays, ultraviolet rays or an electron beam.

In one embodiment, the elastomeric crosslinked organopolysiloxane is obtained by crosslinking addition reaction (A) of a diorganopolysiloxane comprising at least two hydrogens each bonded to a silicon, and (B) of a diorganopolysiloxane having at least two ethylenically unsaturated groups bonded to the silicon, for example in the presence (C) of a platinum catalyst, as described, for example, in European Patent No. EP-A-295886.

For example, the organopolysiloxane may be obtained by reacting a dimethylpolysiloxane having dimethylvinylsiloxy end groups and a methylhydropolysiloxane having trimethylsiloxy end groups, in the presence of a platinum catalyst.

The compound (A) is the base reactant for the formation of elastomeric organopolysiloxane and the crosslinking is carried out by addition reaction of the compound (A) with the compound (B) in the presence of the catalyst (C).

The compound (A) is, for example, an organopolysiloxane having at least two hydrogen atoms bonded to separate silicon atoms in each molecule.

The compound (A) may have any molecular structure, such as a linear chain structure or branched chain structure or a cyclic structure.

The compound (A) may have a viscosity at 25° C. ranging from 1 to 50,000 centistokes, for example, so as to be readily miscible with the compound (B).

The organic groups bonded to the silicon atoms of the compound (A) may be chosen from alkyl groups such as methyl, ethyl, propyl, butyl and octyl; substituted alkyl groups such as 2-phenylethyl, 2-phenylpropyl and 3,3,3-trifluoropropyl; aryl groups such as phenyl, tolyl and xylyl; substituted aryl groups such as phenylethyl; and substituted monovalent hydrocarbon groups such as an epoxy group, a carboxylate ester group and a mercapto group.

The compound (A) may therefore be chosen from, for example, methylhydropolysiloxanes comprising trimethylsiloxy end groups, dimethylsiloxane-methylhydrosiloxane copolymers comprising trimethylsiloxy end groups, and cyclic dimethylsiloxane-methylhydrosiloxane copolymers.

The compound (B) may be, for example, chosen from a diorganopolysiloxane having at least two lower (for example, C₂-C₄) alkenyl groups; the lower alkenyl groups may be chosen from vinyl, allyl, and propenyl groups. These lower alkenyl groups may be situated in any position on the organopolysiloxane molecule but are, in one embodiment, situated at the ends of the organopolysiloxane molecule. The organopolysiloxane (B) may have a structure chosen from branched-chain, linear-chain, cyclic and network structures, for example, the linear-chain structure. The compound (B) may have a viscosity ranging from the liquid state to the rubber state. In one embodiment, the compound (B) has a viscosity of at least 100 centistokes at 25° C.

Besides the aforementioned alkenyl groups, the other organic groups bonded to the silicon atoms in the compound (B) may be, for example, chosen from alkyl groups such as methyl, ethyl, propyl, butyl and octyl; substituted alkyl groups such as 2-phenylethyl, 2-phenylpropyl and 3,3,3-trifluoropropyl; aryl groups such as phenyl, tolyl and xylyl; substituted aryl groups such as phenylethyl; and substituted monovalent hydrocarbon groups, such as an epoxy group, a carboxylate ester group and a mercapto group.

The organopolysiloxanes (B) may be chosen from methylvinylpolysiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethylsiloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxanes comprising dimethylvinylsiloxy end groups, and dimethylsiloxane-methyl(3,3,3-trifluoropropyl)siloxane copolymers comprising dimethylvinylsiloxy end groups.

For example, the elastomeric organopolysiloxane may be obtained by reacting a dimethylpolysiloxane comprising dimethylvinylsiloxy end groups and a methylhydropolysiloxane comprising trimethylsiloxy end groups, in the presence of a platinum catalyst.

In one embodiment, the sum of the number of ethylenic groups per molecule of the compound (B) and the number of hydrogen atoms bonded to silicon atoms per molecule of the compound (A) is at least 5.

In further embodiments, the compound (A) is added in an amount such that the molecular ratio between the total amount of hydrogen atoms bonded to silicon atoms in the compound (A) and the total amount of all of the ethylenically unsaturated groups in the compound (B) is in the range from 1.5/1 to 20/1.

The compound (C) is the catalyst of the crosslinking reaction and is, for example, chosen from chloroplatinic acid, chloroplatinic acid-olefin complexes, chloroplatinic acid-alkenylsiloxane complexes, chloroplatinic acid-diketone complexes, platinum black, and supported platinum.

The catalyst (C) is, for example, added from 0.1 parts to 1000 parts by weight, such as from 1 part to 100 parts by weight, as platinum metal proper, per 1000 parts by weight of the total amount of the compounds (A) and (B).

The elastomer may be a non-emulsifying elastomer.

As used herein, the term “non-emulsifying” means organopolysiloxane elastomers, which do not contain polyoxyalkylene units (e.g., polyoxyethylene or polyoxypropylene).

The elastomeric crosslinked organopolysiloxane particles may be, for example, carried in the form of a gel comprising an elastomeric organopolysiloxane in at least one hydrocarbon oil and/or at least one silicone oil. In these gels, the organopolysiloxane particles may be non-spherical particles.

The elastomeric crosslinked organopolysiloxane particles may also be in the form of a powder, such as a spherical powder.

Non-emulsifying elastomers are described, for example, in U.S. Pat. Nos. 4,970,252, 4,987,169, 5,412,004, 5,654,362 and 5,760,116 and in Japanese Patent No. JP-A-61-194009.

Non-emulsifying elastomers which may be used, for example, inlcude those sold under the names KSG-6, KSG-15, KSG-16, KSG-18, KSG-31, KSG-32, KSG-33, KSG-41, KSG-42, KSG-43 and KSG-44 by the company Shin Etsu, DC9040, DC9041, DC9509, DC9505 and DC9506 by the company Dow Corning, Gransil by the company Grant Industries, and SFE 839 by the company General Electric.

The particles of elastomeric crosslinked organopolysiloxane may also be present in the form of an elastomeric crosslinked organopolysiloxane powder coated with silicone resin, such as with a silsesquioxane resin, as described, for example, in U.S. Pat. No. 5,538,793. Elastomers of this kind are sold under the names KSP-100, KSP-101, KSP-102, KSP-103, KSP-104 and KSP-105 by the company Shin Etsu.

Other elastomeric crosslinked organopolysiloxanes in powder form may be hybrid silicone powders functionalized with fluoroalkyl groups, such as those sold under the name KSP-200 by the company Shin Etsu or hybrid silicone powders functionalized with phenyl groups, such as those sold under the name KSP-300 by the company Shin Etsu.

The particles of elastomeric organopolysiloxane may have a JIS-A hardness of less than or equal to 80 (e.g., ranging from 5 to 80) and further for example, less than or equal to 65 (e.g., ranging from 5 to 65). The JIS-A hardness is measured according to method JIS K 6301 (1995) established by the Japanese Industrial Standards Committee.

For example, the particles of elastomeric organopolysiloxane (in terms of active substance) may have an average size ranging from 0.1 μm to 500 μm, such as ranging from 3 μm to 200 μm, and further for example, ranging from 10 μm to 20 μm. These particles may be spherical, flat or amorphous in shape, and for example, are spherical in shape.

According to one embodiment of the present disclosure, the particles of elastomeric crosslinked organopolysiloxane which may be used are present in the form of an aqueous dispersion, as described in Japanese Patent No. JP-A-10/175816 or U.S. Pat. No. 5,928,660. According to these disclosures, the elastomeric crosslinked organopolysiloxane is obtained by addition reaction and crosslinking reaction, in the presence of a catalyst, such as of the platinum type, of at least:
(a) an organopolysiloxane (i) having at least two vinyl groups in a and w position on the silicone chain, per molecule; and
(b) an organosiloxane (ii) having at least one hydrogen atom bonded to a silicone atom per molecule.

The aqueous dispersion may, for example, be obtained as follows:
(a) mixing an organopolysiloxane (i) having at least two vinyl groups positioned a and w on the silicone chain per molecule and an organosiloxane (ii) having at least one hydrogen atom bonded to a silicone atom per molecule;
(b) adding a catalyst, such as a platinum catalyst;
(b) adding an aqueous phase comprising an emulsifier, in order to form an emulsion; and
(c) polymerizing organopolysiloxane (i) and organosiloxane (ii) in emulsion in the presence of a platinum catalyst.

For example, the organopolysiloxane (i) is chosen from α,ω-dimethylvinylpolydimethylsiloxanes.

The emulsifier may be chosen from nonionic, cationic, and anionic surfactants with an HLB ≧8, and for example, from nonionic surfactants.

The proportion of surfactants may be, for example, from 0.1 parts to 20 parts by weight per 100 parts by weight of the elastomeric organopolysiloxane composition, and further for example, from 0.5 parts to 10 parts by weight (cf. description of Japanese Application No. J-A-10/175816).

After step (c) it may be possible to dry the particles obtained, in order to evaporate some or all of the trapped water from them.

Organopolysiloxane particles dispersed in water may be used; for example, those sold under the names BY 29-122 and BY 29-119 by the company Dow Corning.

The solid particles having a refractive index ranging from 1.2 to 1.45 may be additionally chosen from silica particles having the refractive index. Silicas used may be, for example, from silicas sold under the name Sunsphere H-51 by the company Asahi Glass or Silica Shells by the company Kobo.

The solid particles having a refractive index ranging from 1.2 to 1.45 may also be selected from polymethylsilsesquioxane particles such as those described in European Application No. EP-A-293795 and for example, those sold under the name Tospearl 120A, Tospearl 145A or Tospearl 2000B by the company GE Toshiba Silicones.

The solid particles having a refractive index ranging from 1.2 to 1.45 may be present in the composition according to the present disclosure in an amount ranging from 5% to 80% by weight, relative to the total weight of the composition, such as ranging from 10% to 70% by weight, for example, ranging from 10% to 60% by weight, another example, ranging from 10% to 50% by weight, further for example, ranging from 15% to 50% by weight, such as ranging from 20% to 45% by weight, and yet another example, ranging from 20% to 40% by weight.

The liquid binder present in the composition according to the present disclosure is a liquid having a refractive index ranging from 1.2 to 1.6, such as ranging from 1.2 to 1.5, for example, ranging from 1.25 to 1.45 and further for example, ranging from 1.3 to 1.45.

The liquid binder may be chosen from water, water-miscible organic solvents, oils, and mixtures thereof; for example, the liquid binder may be chosen from water and water-miscible organic solvents, and mixtures thereof.

The water may be chosen from floral water, such as cornflower water and an inorganic water, such as Vittel water, Lucas water and La Roche Posay water, and a spring water.

The composition may comprise water in an amount ranging from 5% to 30% by weight, relative to the total weight of the composition, such as ranging from 10% to 25% by weight, for example, ranging from 15% to 25% by weight and further for example, ranging from 20% to 25% by weight.

The organic solvents which are miscible with water at ambient temperature (25° C.) may be, for example, chosen from monoalcohols having from 2 to 6 carbon atoms, such as ethanol or isopropanol;
polyols having, for example, from 2 to 20 carbon atoms, such as having from 2 to 10 carbon atoms and further for example, having from 2 to 6 carbon atoms, such as glycerol, propylene glycol, butylene glycol, pentylene glycol, hexylene glycol, dipropylene glycol or diethylene glycol;
glycol ethers (having, e.g., from 3 to 16 carbon atoms) such as the C₁-C₄ alkyl ethers of mono-, di- or tripropylene glycol or the C₁-C₄ alkyl ethers of mono-, di- or triethylene glycol;
and mixtures thereof.

The composition according to the present disclosure may comprise a water-miscible organic solvent, such as a polyol, in an amount ranging from 1% to 20% by weight, relative to the total weight of the composition, and for example, ranging from 3% to 15% by weight.

The oils may be chosen from hydrocarbon oils, silicone oils, fluoro oils, and mixtures thereof. The oil may, for example, be chosen from volatile oils.

The hydrocarbon oil is oil generally formed from, or even comprising, atoms of carbon and hydrogen, and possibly of atoms of oxygen or nitrogen, but not comprising silicon or fluorine atoms. It may comprise alcohol, ester, ether, carboxylic acid, amine and/or amide groups.

As defined herein, a volatile oil is any non-aqueous, water-immiscible medium capable of evaporating from the skin or lips in less than one hour, and for example, oils having a vapor pressure, at ambient temperature and atmospheric pressure, ranging from 10⁻³ mmHg to 300 mmHg (0.13 Pa to 40,000 Pa).

The hydrocarbon oils may be, for example, chosen from liquid paraffin and liquid petroleum jelly, mink oil, turtle oil, soya oil, squalane, perhydrosqualene, sweet almond oil, beauty-leaf oil, palm oil, grapeseed oil, sesame oil, corn oil, hydrogenated polyisobutylene (parleam), arara oil, colza oil, sunflower oil, cotton seed oil, apricot oil, castor oil, avocado oil, jojoba oil, olive oil and cereal germ oil; esters of lanolic acid, of oleic acid, of lauric acid and of stearic acid; fatty esters such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate and lactate, 2-diethylhexyl succinate, diisostearyl malate, glyceryl triisostearate and diglyceryl triisostearate; higher fatty acids such as myristic acid, palmitic acid, stearic acid, behenic acid, oleic acid, linoleic acid, linolenic acid and isostearic acid; and higher fatty alcohols such as cetanol and oleyl alcohol, linoleyl alcohol and linolenyl alcohol, isostearyl alcohol and octyldodecanol.

The silicone oils may be, for example, chosen from polydimethylsiloxanes (PDMS), optionally comprising phenyl groups, such as phenyltrimethicones, and optionally substituted by aliphatic and/or aromatic groups, optionally fluorinated, and by functional groups such as hydroxyl, thiol and/or amine groups; polysiloxanes modified with fatty acids, fatty alcohols and polyoxyalkylenes, and fluoro silicones.

In one embodiment, it is possible to use at least one oil, which is volatile at ambient temperature. The at least one oil may be chosen from hydrocarbon oils and silicone oils and may optionally comprise alkyl or alkoxy groups, pendent or at the end of a silicone chain.

Volatile oils which may be used in the present disclosure include, for example, linear or cyclic silicones having 2 to 7 silicon atoms, these silicones optionally comprising alkyl or alkoxy groups having 1 to 10 carbon atoms, for example, octamethylcyclotetrasiloxane, decamethylcyclopentasiloxane, hexadecamethylcyclohexasiloxane, heptamethylhexyltrisiloxane and heptamethyloctyltrisiloxane, and also C₈-C₁₆ isoparaffins such as Isopar® products, Permetyl® products and, for example, isododecane.

The liquid binder of the composition may be present in the composition according to the disclosure in an amount ranging from 5% to 60% by weight, relative to the total weight of the composition, such as ranging from 5% to 50% by weight, for example, ranging from 5% to 45% by weight, such as from 10% to 40% by weight and further for example, ranging from 15% to 35% by weight.

The colorant present in the composition is chosen from reflective particles other than those comprising mica substrates, goniochromatic colorants, photochromic colorants, fluorescent agents, and mixtures thereof.

The composition may comprise as colorant reflective particles other than those comprising mica substrates.

The reflective particles may be chosen from:
particles chosen from natural and synthetic substrates coated at least partially with at least one layer of at least one metal,
particles comprising a synthetic substrate, other than mica, coated at least partially with at least one layer of at least one metal compound, and for example, a metal oxide,
particles formed from a stack of at least two layers of materials having different refractive indices, it being possible for at least one of these layers to be a polymer;
metal particles; and
mixtures thereof.

As disclosed herein, reflective particles mean particles whose size, structure, such as the thickness of the layer or layers of which they are constituted, and their physical and chemical natures, and the surface state, allow them to reflect incident light with sufficient intensity to allow the creation on the surface of the claimed composition, when the latter is applied to the support to be made up, points of brightness which are visible to the naked eye, in other words more luminous points which contrast with their environment, by appearing to shine.

The reflective particles may disrupt the visual perception of the curvature of the made-up support, by tending to prevent lasting visual focusing, the points of brightness being liable to appear or disappear at random when the made-up support and the observer are moving.

The reflective particles used are compatible with a cosmetic use and able to survive in the composition, and for example, do not dissolve, or in any case not dissolve completely, therein.

The reflective particles, for example, have a size which is compatible with the manifestation of a specular reflection of visible light (400 nm-700 nm) of sufficient intensity to create a point of brightness. This size is liable to vary according to the chemical nature of the particles, their shape and their ability to reflect physical light specularly.

Among the reflective particles that may be used herein, some may exhibit a relative difference, Δ, defined by the formula: Δ=[L*_{SCI}−L*_{SCE}/L*_{SCE}], of greater than or equal to 0.25. By comparison, certain nacres that are unsuitable as reflective particles exhibit a coefficient Δ of less than 0.25. In the above formula, L*_{SCI} is the lightness L*, measured by means of a Minolta CM-2002 spectrocolorimeter, in a “specular component included” mode, and L*_{SCE} is the lightness L* measured using the same apparatus in a “specular component excluded” mode. In order to carry out the measurements, a dispersion is produced which contains 5% by weight of the particles under test in a transparent nail varnish of conventional composition (generally nitrocellulose, a resin, and a plasticizer) and a layer 300 μm thick of the composition thus formed is spread in the fluid state onto the black background of a contrast chart.

The SCI/SCE function of the spectrocolorimeter is used with the d/8 geometry to measure L*_{SCI} and L*_{SCE}.

By way of example, the reflective particles of the Reflecks® brand, sold by the company Engelhard, comprising a glass substrate coated with brown iron oxide, a relative difference Δ of more than 0.7 was measured.

The reflective particles may exhibit various forms. These particles may, for example, be in the form of platelets or globes, such as spherical globes.

As used herein, the term “in platelet form” means particles for which the ratio of the largest dimension to the thickness is greater than or equal to 5, or even 10 or, for example, 20. The thickness of particles in platelet form is, for example, from 0.5 μm to 5 μm.

Particles exhibiting a substantially planar outer surface are, for example, suitable, since they are more readily able, if their size, structure and surface condition allow it, to give rise to intense specular reflection. This is referred to as a mirror effect.

For such particles, for example, it is generally the light returned by reflection in a direction forming, with the normal to the reflective surface, the same angle as that formed by the incident light with this normal, that allows these particles to appear as points of brightness, rather than the light scattered in other directions.

The reflective particles may be, for example, non-scattering and non-matt.

It may also be, for example, desirable for the reflective particles not to substantially adversely affect the coloring of the cosmetic composition.

In the present disclosure, suitable reflective particles may be those which allow metallic reflection of the incident light. This is the case, for example, when the reflective particles, irrespective of their form, allow reflection on a layer of a metal, such as of silver, for example. Particles of this kind may be relatively neutral with respect to the color of the composition.

Reflective particles that may be used in the present disclosure, with a metallic or white glint, may, for example, reflect light in all of the components of the visible range without significantly absorbing at least one wavelength. The spectral reflectance of these reflective particles may, for example, be greater than 70% in the 400 nm-700 nm range, and for example, at least 80%, or such as 90% or further for example, 95%.

The light reflected by the reflective particles may be non-iridescent, such as in the case of a metallic glint.

The reflective particles, whatever their shape, may have a multi-layer or non-multilayer structure and, in the case of a multi-layer structure, may have, for example, at least one layer of uniform thickness, such as a layer of a reflective material.

When the reflective particles have a multi-layer structure they may, for example, comprise a natural or synthetic substrate, such as a synthetic substrate at least partly coated with at least one layer of a reflective material.

Irrespective of the shape of the reflective particles, the substrate, when it is synthetic, may be prepared with a shape that promotes the formation of a reflective surface after coating, such as after deposition of a layer of reflective material. The substrate, for example, may have a flat surface and the layer of reflective material a substantially uniform thickness.

The substrate may be made of at least one material and may be solid or hollow. The substrate may be organic or inorganic. The substrate may be natural, although it may, for example, be a synthetic substrate, for the reason indicated above.

The substrate may be chosen from, for example, glasses, ceramics, graphite, metal oxides, aluminas, silicas, silicates, such as aluminosilicates and borosilicates, and synthetic mica.

The reflective material may comprise a layer of metal or of a metal compound, such as a metal oxide.

The metal or metal compound layer may completely cover the substrate, and the metal layer may be at least partly covered with a layer of another material, for example, a transparent material. It may also be, for example, that the metal or metal compound layer coats the substrate completely, directly or indirectly, in other words with interposition of at least one metallic or non-metallic intermediate layer.

The metal may be chosen, for example, from Ag, Au, Cu, Al, Ni, Sn, Mg, Cr, Mo, Ti, Pt, Va, Rb, W, Zn, Ge, Te, Se, and alloys thereof. In one embodiment, the metals are chosen from Ag, Au, Al, Zn, Ni, Mo, Cr, Cu, and alloys thereof (e.g., bronzes and brasses).

In the case, for example, of particles with a substrate coated with silver or gold, the metallic layer may be present in an amount ranging, for example, from 0.1% to. 50% of the total weight of the particles, or such as ranging from 1% to 20%.

Glass particles covered with a metallic layer may have a size ranging, for example, from 10 μm to 300 μm and for example, from 25 μm to 150 μm. Where these particles are in the form of platelets, the thickness may, for example, range from 0.1 μm to 25 μm, such as from 0.5 μm to 10 μm, and further for example, from 0.5 μm to 5 μm. Where these particles are in the form of spheres, they may have a size ranging, for example, from 10 μm to 100 μm.

Glass particles covered with a metallic layer are described, for example, in Japanese Patent Nos. JP-A-09188830, JP-A-1 0158450, JP-A-1 0158541, JP-A-07258460, JP-A-05017710, JP-A-2001-011340 and Japanese Application Nos. JP-A-2002-138010, JP-A-2001-323217, and JP-A-2001-207129.

Still as an example of reflective particles comprising an inorganic substrate coated with a metal layer, mention may also be made of particles comprising a silver-coated borosilicate substrate.

Particles comprising a silver-coated glass substrate in the form of platelets are sold under the name Microglass Metashine REFSX 2025 PS by the company Nippon Sheet Glass. Particles comprising a glass substrate coated with nickel/chromium/molybdenum alloy are sold under the name Crystal Star GF 500 and GF 2525 by this same company.

Irrespective of their shape, the reflective particles may also be chosen from particles comprising a synthetic substrate at least partly coated with at least one layer of at least one metal compound, such as a metal oxide, chosen for example from titanium oxides, such as TiO₂, iron oxides, such as Fe₂O₃, tin oxides, chromium oxides, barium sulphates, and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures and alloys thereof.

Glass particles covered with a layer of metal oxide are described, for example, in Japanese Publication Nos. JP-A-2001-207129 and JP-A-2001-323217, U.S. Pat. Nos. 3,331,699 and 5,436,077, European Patent No. EP-A-912640 and International Patent Application No. WO 02/090448.

Examples that may be mentioned of such particles include, for example, particles comprising a synthetic mica substrate coated with titanium dioxide, or glass particles coated either with brown iron oxide or with titanium oxide or with tin oxide, or a mixture thereof, such as those sold under the brand name Reflecks® by the company Engelhard, or Metashine 1080 R, Metashine MC 1120 RY or Metashine MC 1020 RS by the company Nippon Sheet Glass.

The reflective particles may also be chosen from particles formed by a stack of at least two layers having different refractive indices.

These layers may be polymeric or metallic in nature and may, for example, comprise at least one polymer layer.

Thus, the reflective particles may be particles derived from a multi-layer polymeric film.

Particles of this kind are described, for example, International Publication No. WO 99/36477, and U.S. Pat. Nos. 6,299,979 and 6,387,498.

By way of example of materials which may make up the different layers of the multi-layer structure mention may be made, in a non-limiting list, of the following: polyethylene naphthalate (PEN) and its isomers, for example 2,6-, 1,4-, 1,5-, 2,7- and 2,3-PEN, polyalkylene terephthalates, polyimides, polyetherimides, atactic polystyrenes, polycarbonates, polyalkyl methacrylates and polyalkyl acrylates, syndiotactic polystyrene (sPS), syndiotactic poly-alpha-methylstyrenes, syndiotactic polydichlorostyrene, copolymers and blends of these polystyrenes, cellulose derivatives, polyalkylene polymers, fluropolymers, chloropolymers, polysulphones, polyethersulphones, polyacrylonitriles, polyamides, silicone resins, epoxy resins, polyvinyl acetate, polyetheramides, ionomer resins, elastomers and polyurethanes. Also suitable are copolymers, for example, PEN copolymers (for example, copolymers of 2,6-, 1,4-, 1,5-, 2,7- and/or 2,3-naphthalenedicarboxylic acid or its esters) with (a) terephthalic acid or its esters; (b) isophthalic acid or its esters; (c) phthalic acid or its esters; (d) alkane glycols; (e) cycloalkane glycols (for example cyclohexanedimethanol diol); (f) alkanedicarboxylic acids; and/or (g) cycloalkanedicarboxylic acids, polyalkylene terephthalate copolymers and styrene copolymers. In addition, each individual layer may include blends of at least two of the above polymers or copolymers.

The choice of materials intended to make up the various layers of the multi-layer structure is made, of course, so as to give the particles thus formed a reflective appearance.

Reflective particles comprising a stack of at least two layers of polymers are sold by the company 3M under the name Mirror Glitter. These particles comprise layers of 2,6-PEN and polymethyl methacrylate in a ratio of 80/20 by mass. Particles of this kind are described in U.S. Pat. No. 5,825,643.

The brightness of the reflective particles may also be due, in part, as a variant or additionally, to the reflection of light on a layer of a material of the particle that has a sufficiently large refractive index relative to that of the medium from which the incident light originates.

The reflective metallic particles may be chosen from metal particles such as gold, silver, aluminum, and copper particles.

The cosmetic composition according to the disclosure may comprise reflective particles of other kinds without departing from the scope of the present disclosure.

The colorant present in the composition according to the present disclosure may be chosen from goniochromatic colorants.

A goniochromatic colorant for the purposes of the present disclosure is a colorant which, when the composition comprising it is spread over a support, makes it possible to obtain a color trajectory in the a*b* plane of the CIE 1976 calorimetric space that corresponds to a variation Dh in the hue angle h of at least 20° C. when the angle of observation relative to the normal is varied between 0° and 80°, for an incident light angle of 45°.

The color trajectory may be measured, for example, by an Instrument Systems GON 360 Goniometer spectrogonioreflectometer, after the cosmetic composition has been spread in the fluid state with a thickness of 300 μm, using an automatic spreader, onto an Erichsen type 24/5 contrast chart, the measurement being made on the black background of the chart.

The goniochromatic colorant may be chosen so as to present a relatively large color change with the angle of observation.

The goniochromatic colorant may be chosen, for example, from multi-layer interference structures and liquid-crystal colorants.

A multi-layer structure may comprise, for example, at least two layers, with each layer, independently or otherwise of the other layer (or layers), being made, for example, from at least one material chosen from groups of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers, and combinations thereof.

The multi-layer structure goniochromatic agents are, for example, those described in the following documents: U.S. Pat. No. 3,438,796, European Patent No. EP-A-227423, U.S. Pat. No. 5,135,812, European Patent Nos. EP-A-170439 and EP-A-341002, U.S. Pat. Nos. 4,930,866 and 5,641,719, European Patent Nos. EP-A-472371, EP-A-395410, EP-A-753545, EP-A-768343, EP-A-571836, EP-A-708154 and EP-A-579091, U.S. Pat. No. 5,411,586 and U.S. Pat. No. 5,364,467, International Application No. WO-A-97/39066, German Application No. DE-A-4 225 031, International Application No. WO 9517479 (BASF) and German Application No. DE-A-196 14 637. They are in the form of spangles, with a metallic color.

The multi-layer structures which can be used in the disclosure are, for example, chosen from the following structures: Al/SiO₂/Al/SiO₂/Al; Cr/MgF₂/Al/MgF₂/Al; MOS₂/SiO₂/Al/SiO₂/MoS₂; Fe₂O₃/SiO₂/Al/SiO₂/Fe₂O₃; Fe₂O₃/SiO₂/Fe₂O₃; MoS₂/SiO₂/mica oxide/SiO₂/MOS₂; and Fe₂O₃/SiO₂/mica oxide/SiO₂/Fe₂O₃. Different colors may be obtained according to the thickness of the different layers. Thus, with the structure Fe₂O₃/SiO₂/Al/SiO₂/Fe₂O₃, for example, the color changes from greenish gold to reddish grey for SiO₂ layers of 320 nm to 350 nm; from red to gold for SiO₂ layers of 380 nm to 400 nm; from violet to green for SiO₂ layers of 410 nm to 420 nm; and from copper to red for SiO₂ layers of 430 nm to 440 nm.

Consequently, the multi-layer structure may be generally inorganic or organic. Different colors may be obtained according to the thickness of each of the different layers.

The goniochromatic pigments with a multi-layer interference structure according to the disclosure are, for example, those described in the following documents: U.S. Pat. No. 3,438,796, European Patent No. EP-A-227423, U.S. Pat. No. 5,135,812, European Patent Nos. EP-A-170439 and EP-A-341002, U.S. Pat. Nos. 4,930,866 and 5,641,719, European Patent Nos. EP-A-472371, EP-A-395410, EP-A-753545, EP-A-768343, EP-A-571836, EP-A-708154 and EP-A-579091, U.S. Pat. Nos. 5,411,586 and 5,364,467, International Application No. WO-A-97/39066, German Application No. DE-A-4 225 031, International Application No. WO 9517479 (BASF) and German Application No. DE-A-196 14 637, and combinations thereof. They are present in the form of spangles, with a metallic color.

The goniochromatic pigment with a multi-layer interference structure according to the disclosure may be, for example, chosen from groups comprising the following commercial goniochromatic pigments: Infinite Colors, manufactured or sold by the company Shiseido; Sicopearl Fantastico, manufactured or sold by the company BASF; Colorstream, manufactured or sold by the company Merck; Colorglitter manufactured or sold by the company 3M; Chromaflair, manufactured or sold by the company Flex; Xirallic and Xirona, manufactured or sold by the company Merck; and mixtures thereof.

Goniochromatic agents with a multi-layer structure may be those sold under the name Sicopearl.

The liquid crystal goniochromatic particles suitable for use in the composition according to the disclosure may, for example, be based on a polymer obtainable by polymerizing a mixture of monomers comprising:
a) at least one first monomer A of formula (I)
   Y1-A1-M1-A2-Y2
   wherein
   i) Y1 and Y2, which are identical or different, are each a polymerizable group chosen from acrylate and methacrylate groups, epoxy groups and isocyanate, hydroxyl, vinyl ether (—O—CH═CH₂) and vinyl ester (—CO—O—CH═CH₂) groups, and
   ii) A1 and A2, which are identical or different, are each a group of formula —CₙH₂ₙ—, wherein n is an integer ranging from 0 to 20, it being possible for at least one methylene group of the —CₙH₂ₙ— group to be replaced by at least one oxygen atom, and
   iii) Ml is a group of general formula (I′)
      —R₁—X₁—R₂—X₂—R₃—X₃—R₄—, wherein R₁, R₂, R₃ and R₄, which are identical or different, are chosen from divalent groups of —O—, —COO—, —CONH—, —CO—, —S—, —C≡C—, —CH═CH—, —N═N— and —N═N(O)—, it being possible for —R₂—X₂—R₃— or —R₂—X₂— or —R₂—X₂—R₃—X₃— also to be a single covalent bond, and X₁, X₂ and X₃, which are identical or different, are chosen from 1,4-phenylene groups, 1,4-cyclohexylene groups, arylene and heteroarylene groups having an aryl nucleus comprising from 6 to 10 atoms which are optionally substituted by B1 and/or B2 and/or B3, the heteroarylene comprising from 1 to 3 heteroatoms chosen from the O, N and S atoms, and cycloalkylene groups having from 3 to 10 carbon atoms which are optionally substituted by —B1 and/or —B2 and/or —B3, —B1, —B2 and —B3, which are identical or different, are chosen from C₁-C₂₀ alkyl, C₁-C₂₀ alkoxy, C₁-C₂₀ alkylthio, (C₁-C₂₀)alkyl-carbonyl, (C₁-C₂₀)alkoxy-carbonyl, (C₁-C₂₀)alkyl-thiocarbonyl, —OH, —F, —Cl, —Br, —I, —CN, —NO₂, formyl and acetyl groups and alkyl, alkoxy and alkylthio groups having from 1 to 20 carbon atoms which are interrupted by at least one oxygen atom, optionally at least one sulfur atom and optionally, at leas one ester group, and
b) at least one chiral second monomer B of formula (II)
   V1-A′1-W1-Z-W2-A′2-V2,
   wherein
   i) V1 and V2, which are identical or different, are groups chosen from acrylate and methacrylate groups; epoxy groups; vinyl ether and vinyl ester groups; isocyanate groups; C₁-C₂₀ alkyl, C₁-C₂₀ alkoxy, C₁-C₂₀ alkylthio, (C₁-C₂₀)alkoxy-carbonyl, (C₁-C₂₀)alkyl-thiocarbonyl, —OH, —F, —Cl, —Br, —I, —CN, —NO₂, formyl, acetyl and alkyl, alkoxy and alkylthio groups, having from 1 to 20 carbon atoms and interrupted by at least one oxygen atom, at least one sulfur atom, or at least one ester group (—CO—O—); and at least V1 or V2 is a polymerizable group chosen from acrylate and methacrylate groups, epoxy groups and isocyanate, hydroxyl, vinyl ether (—O—CH═CH₂) and vinyl ester (—CO—O—CH═CH₂) groups,
   ii) A′1 and A′2, which are identical or different, are groups of formula —CₙH₂ₙ—, wherein n is an integer ranging from 0 to 20, wherein at least one methylene group of the CₙH₂ₙ group may be replaced by at least one oxygen atom, and
   iii) W1 and W2 are divalent groups of general formula
      R′₁—X′₁—R′₂—X′₂—R′₃—,
      wherein R′₁, R′₂ and R′₃, which are identical or different, are divalent groups chosen from —O—, —COO—, —CONH—, —CO—, —S—, —C≡C—, —CH═CH—, —N═N— and —N═N(O)— groups; and R′₁, R′₂, R′₃ or R′₂—X′₂ may be a single covalent bond, and X′₁ and X′₂, which are identical or different, are groups chosen from 1,4-phenylene groups, 1,4-cyclohexylene groups, arylene and heteroarylene groups having an aryl nucleus comprising from 6 to 10 atoms which are optionally substituted by B′1 and/or B′2 and/or B′3, the heteroarylene comprising from 1 to 3 heteroatoms chosen from O, N and S atoms, and cycloalkylene groups having from 3 to 10 carbon atoms which are optionally substituted by —B′1 and/or —B′2 and/or —B′3,—B′1, —B′2 and —B′3, which are identical or different, are chosen from C₁-C₂₀ alkyl, C₁-C₂₀ alkoxy, C₁-C₂₀ alkylthio, (C₁-C₂₀)alkyl-carbonyl, (C₁-C₂₀)alkoxy-carbonyl, (C₁-C₂₀)alkyl-thiocarbonyl, —OH, —F, —Cl, —Br, —I, —CN, —NO₂, formyl and acetyl groups and alkyl, alkoxy and alkylthio groups having from 1 to 20 carbon atoms which are interrupted by at least one oxygen atom, optionally at least one sulfur atom, and optionally, at least one ester group, and Z is a chiral divalent group comprising at least 4 carbon atoms, such as from 4 to 20 carbon atoms and for example, from 4 to 10 carbon atoms (the chiral divalent group comprising at least one asymmetric carbon, such as one or two asymmetric carbons and for example, two asymmetric carbons), and further for example, a chiral divalent group originating from the groups chosen from dianhydrohexitols, hexoses, pentoses, binaphthyl derivatives (binaphthyl groups), biphenyl derivatives (biphenyl groups), tartaric acid derivatives and glycols which are optically active.

In one embodiment, the liquid crystal polymer is obtained by polymerizing a mixture of monomers comprising:
a) at least one first monomer A of formula (I) Y1-A1-M1-A2-Y2
   wherein
   i) Y1 and Y2, which are identical or different, are chosen from acrylate and methacrylate groups, for example acrylate groups;
   ii) A1 and A2, which are identical or different, are groups of formula —CₙH₂ₙ—, wherein n is an integer ranging from 1 to 20, such as ranging from 2 to 6 and for example, equal to 4;
   iii) M1 is a group of general formula (I′)
      —R₁—X₁—R₂—X₂—R₃—X₃—R₄—, wherein R₁ and R₄ are —O— atoms, R₂ and R₃ are —COO— groups and X₁, X₂ and X₃ are 1,4-phenylene groups, the carbonyl group —CO— respectively of R₂ and of R₃ being bonded respectively to the X₁ group and to the X₃ group, and
b) at least one chiral second monomer B of formula (II) V1-W1-Z-W2-V2, wherein
   i) V1 is chosen from acrylate and methacrylate groups, and for example, an acrylate group, and V2 is chosen from C₁-C₂₀ alkyl groups, C₁-C₂₀ alkoxy, (C₁-C₂₀)alkoxy-carbonyl and —OH groups and, for example, is a C₁-C₂₀ alkoxy group, such as a C₁-C₄ alkoxy group and, further for example, a methoxy group;
   ii) W1 is a divalent group of formula —X′1-CO—O—, W2 is a divalent group of formula —O—CO—X′1-, wherein formulae X′1 is a 1,4-phenylene group, and Z is a chiral group comprising two bonds, originating from the dianhydrohexitol group, such as a divalent radical of formula:

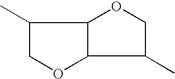

The mixture of monomers, for example, comprises from 70% to 99% by weight of monomer A and from 1% to 30% by weight of monomer B, relative to the total weight of monomer A and monomer B, and further for example, comprises from 90% to 95% by weight of monomer A and from 5% to 10% by weight of monomer B.

The concentration of the polymerizable groups present in the mixture of monomer A and monomer B (polymerizable groups Y1 and Y2 of the monomer A and polymerizable groups V1 and V2 of the monomer B), for example, ranges from 3.2 mmol/g to 15 mmol/g.

According to one embodiment of the disclosure, the liquid crystal polymer is such that the mixture of monomer A and of monomer B comprises polymerizable groups, at least 90% of which are present in monomers having at least two polymerizable groups, in a concentration ranging from 3.2 mmol/g to 15 mmol/g.

For example, the liquid crystal polymer generally comprises a mixture of the monomers A and B defined above.

The liquid crystal polymer, for example, exhibits a helical pitch of greater than 450 nm, such as ranging from 455 nm to 5,000 nm, for example, ranging from 455 nm to 1,000 nm and further for example, ranging from 455 nm to 650 nm.

The monomer A may possess a weight-average molecular weight ranging from 150 to 800 and for example, ranging from 460 to 625. The monomer A is, for example, an unsubstituted hydroquinone dibenzoate derivative.

The monomer B may have a weight-average molecular weight ranging from 500 to 1,000 and for example, ranging from 500 to 700.

The liquid crystal polymer may possess a weight-average molecular weight of less than 625.

The liquid crystal polymer defined above can be prepared from the monomer mixture described above according to the processes known in the state of the art, such as those disclosed in U.S. Pat. Nos. 5,362,315 and 5,807,497.

The polymerization of the oriented mixture of monomers may be carried out in a way already known, for example, in the free radical fashion with the use of commercial thermal initiators, using electron beams or UV light in combination with commercial photo-initiators, or else by addition or condensation reactions.

The crosslinking of the mixtures of monomers, in the chiral structural state, is, for example, carried out by a polyreaction, which, according to the type of the polymerizable, polycondensable or polyadditionable groups, takes place in the form of a free-radical, ionic or metal-catalysed polymerization or of a polycondensation reaction or of a polyaddition reaction.

The free-radical polymerization may be initiated by corresponding initiators or by UV radiation, using commercial photoinitiators, or by high-energy radiation, such as electron radiation. For example, an advantage of the thermal polymerization of radicals or of the polymerization via curing with electron beams may be that a photoprotectant, such as a UV (UVA) absorber or free-radical scavengers (HALSs), may also be added to the polymerizable mixture to stabilize the resulting films or pigments in the face of UV light, for example,.for exterior applications, without resulting in losses as regards the polymerization conversion, as is the case with UV curing, because of the effect of screening of the photoinitiator by UVA radiation. Thus, there may be no decrease in the crosslinking density.

If the films are cured using peroxide or by electron radiation, then the mixture of monomers, for example, comprises commercial photoprotectants, such as UV absorbers or free-radical scavengers, in an overall concentration ranging from 0.5% to 5% by weight.

In addition to the photostabilizers, the mixtures of monomers may also comprise other customary additives targeted at inhibiting oxidation or at inhibiting polymerization or additives targeted at improving the rheological properties. Furthermore, absorbent fillers, such as pigments or soot, and also dyes or fluorescent pigments may be present.

The film obtained after the polymerization is subsequently milled into particles, such as in the form of platelets.

For example, the particles of liquid crystal polymer have a larger size ranging from 1 μm to 3 mm and, further for example, ranging from 30 μm to 500 μm. These particles may be, for example, in the form of platelets.

The particles may be separated (i.e., sorted) by a process with selectivity for the grain size.

Such polymers and their particles are disclosed in European Patent No. EP-A-1 046 692.

Particles of liquid crystal polymer that may be used, for example, are those known under the CTFA name Polyacrylate-4 and sold under the names “Helicone® HC Sapphire”, “Helicone® HC Scarabeus”, “Helicone® HC Jade”, “Helicone® HC Maple”, “Helicone® HC XL Sapphire”, “Helicone® HC XL Scarabeus”, “Helicone® HC XL Jade,” and “Helicone® HC XL Maple” by Wacker.

The colorant present in the composition may also be chosen from photochromic coloring agents.

As defined herein, a photochromic coloring agent is an agent which has the property of changing color when it is lit by ultraviolet light and of regaining its initial color when it is no longer lit by the said light, or else of passing from an uncolored state to a colored state, and vice versa. For example, these agents exhibits different colors depending on whether it is lit with natural light or artificial light.

The photochromic agents, which may be used in the disclosure, are, for example, those described in Japanese Patent No. JP-A-09/165532, European Patent No. EP-A-709728, Japanese Patent Nos. JP-A-07/258580 and JP-A-07/223816, European Patent No. EP-A-624553, Japanese Patent Nos. JP-A-08/337422 and JP-A-07/025617, and European Patent No. EP-A-359909.

For example, the photochromic coloring agents which may be used in the disclosure may be chosen from spiro-oxazines and derivatives thereof, such as spiroindolino-naphthooxazines; spironaphthoxazines; naphthopyran and its derivatives; spiropyrans, such as indolinospirobenzopyrans; nitrobenzylpyridines; spirolans; titanium oxide and zinc oxide doped with iron. By way of example, mention may be made of photochromic agents of the type derived from naphthopyran, sold by the company PPG under the references Photosol 5-68 Photochromic Dye, Photosol 7-49 Photochromic Dye, Photosol 7-106 Photochromic Dye, Photosol 0265 Photochromic Dye, Photosol 0272 Photochromic Dye, these agents exhibiting two different colors depending on whether they are excited or not by UV. Other photochromic coloring agents which may be used herein may be chosen from doped aluminosilicates, doped, for example, with the groups S, Se, SO₄²⁻, WO₄²⁻ or OH, and with metal ions, such as Fe, Cr, Mn, Co and Ni ions, such as those described in European Patent No. EP-A-847751.

The colorant present in the composition comprises fluorescent agents (or substances).

The fluorescent agents are well known to the skilled artisan. They may be pigments or dyes. For example, pigments may be chosen from organic and inorganic particles which are insoluble in the composition. Dyes may be, for example, chemical compounds dissolved in the composition. The dyes may be water-soluble or fat-soluble. Fluorescent substances are, for example, described in “Luminescent materials (fluorescent daylight)”, Encyclopedia of Chemical Technology, Kirk-Othmer, vol 14, p. 546-569, 3rd edition, 1981, Wiley.

As defined herein, a fluorescent agent is a substance, which, under the effect of ultraviolet rays and/or of visible light, re-emits in the visible range the portion of light that it has absorbed under the same color as that which it reflects naturally. The color reflected naturally is thus reinforced by the re-emitted color, and appears extremely bright.

Fluorescent agents may be made of inorganic fluorescent substances such as those described in Japanese Patent No. JP 05-117127 and, for example, inorganic fluorescent substances based on zinc oxide.

Fluorescent agents useful herein may also be chosen from organic fluorescent substances, such as daylight-fluorescent pigments; these pigments are generally manufactured from fluorescent dyes, which are dissolved beforehand in a support resin to give a solid solution, which is subsequently ground to a powder of resin particles exhibiting fluorescent properties. The preparation of such fluorescent pigments is described in European Patent No. EP 0 370 470, and U.S. Pat. Nos. 2,851,424, 3,711,604, 3,856,550 and 2,938,878.

Fluorescent pigments suitable for use herein may thus be chosen from, for example, colored polyamide resins, formaldehyde/benzoguanamine resins, melamine/formaldehyde/sulphonamide resins, colored aminotriazine/formaldehyde/sulphonamide cocondensates, metallized polyester flakes, and mixtures thereof. These fluorescent pigments may also be present in the form of aqueous dispersions of fluorescent pigments.

For example, fluorescent pigments suitable for the present disclosure may be chosen from the pink-colored fluorescent aminotriazine/formaldehyde/sulphonamide cocondensate with an average particle size of 3-4 microns that is sold under the trade name “Fiesta Astral Pink FEX-1”, the blue-colored amino-triazine/formaldehyde/sulphonamide cocondensate with an average particle size of 3-4.5 microns that is sold under the trade name “Fiesta Comet Blue FTX-60” by the company Swada, the benzoguanamine/formaldehyde resin, covered with formaldehyde/urea resin and colored yellow, sold under the trade name “FB-205 Yellow”, the benzoguanamine/formaldehyde resin, covered with formaldehyde/urea resin and colored red, sold under the trade name “FB-400 Orange Red” by the company UK Seung Chemical, and the orange-colored polyamide resin sold under the trade name “Flare 911 Orange 4” by the company Sterling Industrial Colors.

The fluorescent agents may also be chosen from optical brighteners, which may be white organic fluorescent substances.

Optical brighteners are compounds thatare well known to the skilled artisan. Such compounds are described in “Fluorescent Whitening Agent, Encyclopedia of Chemical Technology, Kirk-Othmer”, vol 11, pp. 227-241, 4th edition, 1994, Wiley.

Their use in cosmetology as optical skin-whitening agents may be advantageous, for example, because they are composed of chemical compounds endowed with fluorescence properties, which absorb in the ultraviolet (maximum absorption at a wavelength of less than 400 nm) and re-emit the energy by fluorescence for a wavelength of between 380 nm and 830 nm.

These optical brighteners may be defined, for example, as compounds which absorb generally in the UVA ranging from 300 nm to 390 nm and re-emit generally from 400 nm to 525 nm.

Their lightening effect resides, for example, in an emission of energy ranging from 400 nm to 480 nm, which corresponds to an emission in the blue part of the visible region, which contributes to lightening the skin visually when the said emission takes place on the skin.

Optical brighteners known, for example, include stilbene derivatives, such as polystyrylstilbenes and triazinylstilbenes, coumarin derivatives, such as hydroxycoumarins and aminocourmarins, oxazole, benzoxazole, imidazole, triazole and pyrazoline derivatives, pyrene derivatives, porphyrin derivatives, and mixtures thereof.

Such compounds are widespread in commerce. They include, for example, the following derivatives chosen from:
the stilbene derivative of naphthotriazole, sold under the trade name Tinopal GS, the disodium 4,4′-distyrylbiphenylsulphonate (CTFA name: disodium distyrylbiphenyl disulphonate) sold under the trade name Tinopal CBS-X, the cationic aminocoumarin derivative sold under the trade name Tinopal SWN CONC., the sodium 4,4′-bis[(4,6-dianilino-1,3,5-triazin-2- yl)amino]stilbene-2,2′-disulphonate sold under the trade name Tinopal SOP, the 4,4′-bis[(4-anilino-6-bis(2-hydroxyethyl)amino-1,3,5-triazin-2-yl)amino]stilbene.-2,2′-disulphonic acid sold under the trade name Tinopal UNPA-GX, the 4,4′-bis[(4-anilino-6-morpholine-1,3,5-triazin-2-yl)amino]stilbene sold under the trade name Tinopal AMS-GX, and the disodium 4,4′-bis[(4-anilino-6-(2-hydroxyethyl)methyl amino-1,3,5-triazin-2-yl)amino]stilbene-2,2′-disulphonate sold under the trade name Tinopal 5BM-GX, all by the company CIBA Specialites Chimiques;
the 2,5-thiophenediylbis(5-tert-butyl-1,3-benzoxazole) sold under the trade name Uvitex OB by the company CIBA;
the anionic derivative of diaminostilbene in dispersion in water, sold under the trade name Leucophor BSB liquide by the company Clariant; and
mixtures thereof.

The optical brighteners which may be used in the present disclosure may also be in the form of copolymers, for example, acrylates and/or methacrylates, grafted with optical brightener groups as described in French Application. No. FR 99 10942.

For example, optical brighteners suitable for use herein are chosen from disodium 4,4′-distyrylbiphenylsulphonate, sodium 4,4′-bis[(4,6-dianilino-1,3,5-triazin-2-yl)amino]stilbene-2,2′-disulphonate, 2,5-thiophenediyl-bis(5-tert-butyl-1,3 benzoxaxole), and mixtures thereof.

The colorant may be present in the composition according to the present disclosure in an amount ranging from 1% to 80% by weight, relative to the total weight of the composition, such as ranging from 1% to 70% by weight, for example ranging from 1% to 60% by weight, such as ranging from 5% to 50% by weight, further for example, ranging from 5% to 40% by weight, and such as, ranging from 5% to 30% by weight.

The composition according to the present disclosure may comprise an additional pulverulent colorant other than the colorants described above.

The additional pulverulent colorant may be chosen from pigments and nacres other than the colorants described above.

As used herein, the term “pigments” should be understood to mean white or colored, organic or inorganic particles, of any shape, which are insoluble in the physiological medium and are intended for coloring the composition.

As used herein, the term “nacres” should be understood to mean iridescent particles, of any shape, which, for example, are produced by certain molluscs in their shell, or else are synthesized.

The pigments may be present in the composition in an amount ranging from 0.5% to 30% by weight, relative to the weight of the composition, such as ranging from 0.5% to 20% by weight.

The pigments may be white or colored, inorganic, and/or organic. Among inorganic pigments mention may be made, for example, of titanium dioxide, optionally surface-treated, cerium oxide or zirconium oxide, and also zinc oxide, iron oxides (black, yellow or red) or chromium oxide, manganese violet, ultramarine blue, chromium hydrate and Prussian blue.

Among organic pigments, mention may be made of carbon black, D&C pigments, and barium, strontium, calcium and aluminium lakes based on cochineal carmine.

The nacres may be present in the composition in an amount ranging from 0.5% to 50% by weight, relative to the total weight of the composition, such as ranging from 0.5% to 30% by weight.

The nacreous pigments may be chosen from white nacreous pigments such as mica covered with titanium and with bismuth oxichloride, colored nacreous pigments such as titanium mica covered with iron oxides, titanium mica covered with, for example, Prussian blue and chromium oxide, titanium mica covered with an organic pigment of the aforementioned type, and nacreous pigments based on bismuth oxychloride.

The composition according to the present disclosure may comprise additional fillers other than the aforementioned solid particles having a refractive index ranging from 1.2 to 1.45.

As used herein, the term “fillers” should be understood to mean colorless or white, inorganic or synthetic particles, of any shape, which are insoluble in the medium of the composition irrespective of the temperature at which the composition is manufactured.

The fillers may be inorganic or organic and of any shape, platelet-shaped, spherical and oblong, irrespective of the crystallographic morphology (for example, leaflet, cubic, hexagonal, orthorhombic, etc.). Mention may be made of talc, mica, sericite, silica particles having a refractive index of more than 1.45, kaolin, polyamide (Nylon®) powders, poly-β-alanine powders, polyethylene powders, lauroyllysine, starch, boron nitride, hollow polymeric microspheres, such as those of polyvinylidene chlorides/acrylonitrile, for example, Expancel® (Nobel Industrie), acrylic acid copolymer powders, polymethyl methacrylate particles, ethylene glycol dimethacrylate-lauryl methacrylate copolymer powders sold under the name POLYTRAP® 6603 adsorber by the company RP Scherer, precipitated calcium carbonate, magnesium carbonate and magnesium hydrocarbonate, hydroxyapatite, ceramic microcapsules, metal soaps derived from organic carboxylic acids having from 8 to 22 carbon atoms, such as from 12 to 18 carbon atoms, for example zinc stearate, magnesium stearate or lithium stearate, zinc laurate and magnesium myristate; and mixtures thereof.

The additional fillers are, for example, chosen from mica, boron nitride, silica particles having a refractive index of more than 1.45, polyamide (e.g., Nylon®) powders, polyethylene powders, ethylene glycol dimethacrylate-lauryl methacrylate copolymer powders, polymethyl methacrylate particles, and mixtures thereof.

The additional fillers may be present in the composition in an amount such that the optical effect of the particular colorants is unaffected. For example, the fillers may be present in an amount ranging from 0.5% to 50% by weight, relative to the total weight of the composition, such as ranging from 0.5% to 30% by weight and further for example, ranging from 0.5% to 20% by weight.

In one embodiment, the composition according to the present disclosure may include a total amount of additional particles, other than the solid particles having a refractive index ranging from 1.2 to 1.45, (i.e. colorant+additional filler+additional pulverulent colorant) ranging from 20% to 80% by weight relative to the total weight of the composition, such as ranging from 30% to 70% by weight, for example ranging from 35% to 65% by weight and, further for example, ranging from 40% to 60% by weight.

For example, the particles having a refractive index ranging from 1.2 to 1.45 and the other additional particles, other than the solid particles having a refractive index ranging from 1.2 to 1.45 (i.e. colorant+additional filler+additional pulverulent colorant) are present in the composition according to the present disclosure in amounts such that the crosslinked organopolysiloxane particles (i.e., the solid particles)/additional particles' weight ratio, i.e., colorant, filler and when present, additional pulverulent colorant, is from 0.4/1 to 2.5/1, such as from 0.4/1 to 2/1, for example, from 0.4/1 to 1.5/1, such as, from 0.4/1 to 1.3/1, and, further for example, from 0.6/1 to 1.3/1.

The composition may comprise other usual cosmetic ingredients, which may be chosen, for example, from antioxidants, perfumes, preservatives, neutralizing agents, fibers, surfactants, waxes, sunscreens, vitamins, moisturizers, self-tanning compounds, and anti-wrinkle actives.

The skilled artisan will of course take care to select this or these possible additional compounds and/or their amount, in such a way that the advantageous properties of the composition according to the disclosure are not, or not substantially, adversely affected by the intended addition.

The makeup composition according to the disclosure may be present in a variety of formulation forms chosen from, for example, a loose powder, a compact powder, a cream, a solid composition (for example, of the hot-cast type), an anhydrous gel, an aqueous gel, and a stick. The makeup composition is, for example, a composition used without rinsing.

In one embodiment, the composition according to the disclosure may be a powderous composition. A powderous composition is a composition comprising a pulverulent phase and having a texture which may be in the form of a loose powder, a solid composition (compact powder or pressed powder) or in the form of a pasty composition. The powderous composition is not a liquid composition (in the sense of a composition which flows under its own weight at ambient temperature (25° C.)). For example, the powderous composition according to the disclosure may be a solid composition.

A solid composition is a composition which does not flow under its own weight at ambient temperature (25° C.) within one hour.

According to one embodiment, the present disclosure likewise provides a powderous makeup composition comprising solid particles of elastomeric crosslinked organopolysiloxane, water, and a colorant chosen from reflective particles other than those comprising mica substrates, goniochromatic coloring agents, photochromic coloring agents, fluorescent agents, and mixtures thereof.

Other than in the operation examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that numerical ranges and parameters setting forth the broad scope of the disclosure are approximations, the numerical values set forth in the specific examples are reported to as precisely as possible. Any numerical value, however, inherently contains certain errors necessarily resulting from the standard deviation found in their respective testing measurements.

The disclosure is illustrated in more detail by the examples described below, but not limited thereby.

### EXAMPLE 1

An eye shadow was prepared to have following composition:

| | | |
|---|---|---|
| Elastomeric crosslinked polydimethylsiloxane | 45 | g, i.e., |
| particles in an aqueous dispersion comprising | 28.35 | g of ai* |
| 63% of crosslinked polymer | | |
| (BY 29-119 from Dow Corning) | | |
| Silver-coated glass flakes | 20 | g |
| (Metashine ME 2040 PS from | | |
| Nippon Sheet Glass) | | |
| Mica-titanium oxide | 26.6 | g |
| (Flamenco Blue from Engelhard) | | |
| Prussian blue mica-titanium oxide | 2.4 | g |
| (Colorona Dark Blue from Merck) | | |
| Glycerol | 5 | g |
| Preservative | 1 | g |

*ai = active ingredient

After the ingredients were mixed, the composition was either sieved to give a loose powder or was packed in a dish by pressing to give a compact product. Following application to the skin, the product imparted a makeup effect, which exhibited a bright silvery metallic appearance.

### EXAMPLE 2

A blusher was prepared to have the following composition:

| | | |
|---|---|---|
| Elastomeric crosslinked polydimethylsiloxane | 45 | g, i.e. |
| particles in an aqueous dispersion comprising 63% | 28.35 | g ai* |
| of crosslinked polymer (BY 29-119 from Dow Corning) | | |
| Multilayer-structure interference pigment | 20 | g |
| (Sicopearl fantastico pink from BASF) | | |
| Titanium oxide-brown iron oxide-mica | 29 | g |
| (Flamenco Satin Red 460M from Engelhard) | | |
| Glycerol | 5 | g |
| Preservative | 1 | g |

*ai = active ingredient

After the ingredients were mixed, the composition was either sieved to give a loose powder or was packed in a dish by pressing to give a compact product. Following application to the skin, the product imparted a makeup effect, which exhibited intense colored glints. Depending on the angle at which the makeup was observed, a marked change in color from pink to gold was perceived.

### EXAMPLE 3

An eye shadow was prepared to have the following composition:

| | | |
|---|---|---|
| Elastomeric crosslinked polydimethylsiloxane | 28 | g |
| particles (DC9506 from Dow Corning) | | |
| Water | 17 | g |
| Silver-coated glass flakes | 49 | g |
| (Metashine ME 2040 PS from Nippon Sheet Glass) | | |
| Glycerol | 5 | g |
| Preservative | 1 | g |

After the ingredients were mixed, the composition was either sieved to give a loose powder or was packed in a dish by pressing to give a compact product. Following application to the skin, the product imparted a makeup effect, which exhibited a bright silvery metallic appearance.

### EXAMPLE 4

An eye shadow was prepared to have the following composition:

| | | |
|---|---|---|
| Elastomeric crosslinked polydimethylsiloxane | 28 | g |
| particles (DC9506 from Dow Corning) | | |
| Water | 17 | g |
| Multilayer-structure interference pigment | 49 | g |
| (Sicopearl ® fantastico pink from BASF) | | |
| Glycerol | 5 | g |
| Preservative | 1 | g |

After the ingredients were mixed, the composition was either sieved to give a loose powder or was packed in a dish by pressing to give a compact product. Following application to the skin, the product imparted a makeup effect, which exhibited intense colored glints. Depending on the angle at which the makeup was observed, a marked change in color from pink to gold was perceived.

### EXAMPLE 5

A foundation was prepared to have the following composition:

| | | |
|---|---|---|
| Elastomeric crosslinked polydimethylsiloxane | 50 | g, i.e. |
| particles in an aqueous dispersion | 31.5 | g ai* |
| comprising 63% of crosslinked polymer | | |
| (BY 29-119 from Dow Corning) | | |
| Photochromic colorant (Photolight Pearl | 10 | g |
| Red-80 from Nihon Kokken) | | |
| Pigments (iron oxide, titanium dioxide) | 5 | g |
| Nylon powder (Orgasol ® 2002 extra D NAT | 20 | g |
| COS from ATOFINA) | | |
| Glycerol | 5 | g |
| Propylene glycol | 5 | g |
| Ethylene glycol dimethacrylate-lauryl | 4 | g |
| methacrylate copolymer powder, sold under | | |
| the name POLYTRAP ® 6603 adsorber by RP | | |
| Scherer | | |
| Preservative | 1 | g |

*ai = active ingredient

After the ingredients were mixed, the composition was either sieved to give a loose powder or was packed in a dish by pressing to give a compact product. Following application to the skin, the product imparted a makeup effect whose hue changed under the effect of UV excitation (for example, under a UV lamp).

### EXAMPLE 6

An eye shadow was prepared to have the following composition:

| | | |
|---|---|---|
| Elastomeric crosslinked polydimethylsiloxane | 40 | g, i.e. |
| particles in an aqueous dispersion | 25.2 | g ai* |
| comprising 63% of crosslinked polymer | | |
| (BY 29-119 from Dow Corning) | | |
| Glycerol | 5 | g |
| Butylene glycol | 5 | g |
| Goniochromatic liquid-crystal pigments (Helicone ® | 20 | g |
| HC Scarabeus - from the company Wacker) | | |
| Mica-titanium oxide (Flamenco Blue from Engelhard) | 15 | g |
| Pigments (black iron oxide) | 5 | g |
| Nylon powder (Orgasol ® 2002 extra D NAT COS | 9 | g |
| from ATOFINA) | | |
| Preservative | 1 | g |

*ai = active ingredient

After the ingredients were mixed, the composition was either sieved to give a loose powder or was packed in a dish by pressing to give a compact product. Following application to the skin, the product imparted a makeup effect, which exhibited intense colored glints.

### EXAMPLE 7

A makeup powder was prepared to have the following composition:

| | | |
|---|---|---|
| Elastomeric crosslinked polydimethylsiloxane | 45 | g, i.e. |
| particles in an aqueous dispersion | 28.35 | g ai* |
| comprising 63% of crosslinked polymer | | |
| (BY 29-119 from Dow Corning) | | |
| Glycerol | 5 | g |
| Cyclopentadimethylsiloxane | 5 | g |
| Sodium and calcium borosilicate platelets | 24 | g |
| coated with titanium dioxide and tin | | |
| dioxide (92.5/7/0.5) (REFLECKS VISIONS OF | | |
| VIOLET G 530 L - Engelhard) | | |
| Mica-titanium oxide (Flamenco Blue from Engelhard) | 15 | g |
| Pigments (black iron oxide) | 5 | g |
| Preservative | 1 | g |

*ai = active ingredient

After the ingredients were mixed, the composition was either sieved to give a loose powder or was packed in a dish by pressing to give a compact product. Following application to the skin, the product imparted a makeup effect, which exhibited intense colored glints.

1. A composition comprising
elastomeric crosslinked organopolysiloxane particles having a refractive index ranging from 1.2 to 1.45;
a liquid binder having a refractive index ranging from 1.2 to 1.6; and
a colorant chosen from reflective particles other than those comprising mica substrates; goniochromatic coloring agents; photochromic coloring agents; fluorescent agents; and mixtures thereof.

2. The composition according to claim 1, wherein the refractive index of the elastomeric crosslinked organopolysiloxane particles ranges from 1.25 to 1.45.

3. The composition according to claim 1, wherein the elastomeric crosslinked organopolysiloxane particles are chosen from those obtained by:
crosslinking addition reaction of a diorganosiloxane comprising at least one hydrogen bonded to a silicon and a diorganopolysiloxane having ethylenically unsaturated groups bonded to the silicon;
dehydrogenation crosslinking condensation reaction between a diorganopolysiloxane having hydroxyl end groups and a diorganopolysiloxane comprising at least one hydrogen bonded to a silicon;
crosslinking condensation reaction of a diorganopolysiloxane having hydroxyl end groups and a hydrolyzable organopolysiloxane;
thermal crosslinking of organopolysiloxane; and
crosslinking of organopolysiloxane by high-energy radiation.

4. The composition according to claim 3, wherein the elastomeric crosslinked organopolysiloxane particles are obtained by crosslinking addition reaction (A) of a diorganopolysiloxane comprising at least two hydrogens each bonded to a silicon and (B) of a diorganopolysiloxane having at least two ethylenically unsaturated groups bonded to the silicon.

5. The composition according to claim 4, wherein the elastomeric crosslinked organopolysiloxane particles are obtained in the presence of a platinum catalyst.

6. The composition according to claim 1, wherein the elastomeric crosslinked organopolysiloxane particles are obtained by reacting a dimethylpolysiloxane having dimethylvinylsiloxy end groups and a methylhydropolysiloxane having trimethylsiloxy end groups in the presence of a platinum catalyst.

7. The composition according to claim 1, wherein the elastomeric crosslinked organopolysiloxane particles are non-emulsifying elastomers.

8. The composition according to claim 1, wherein the elastomeric crosslinked organopolysiloxane particles having a refractive index ranging from 1.2 to 1.45 are present in an amount ranging from 5% to 80% by weight, relative to the total weight of the composition.

9. The composition according to claim 1, wherein the liquid binder has a refractive index ranging from 1.2 to 1.5.

10. The composition according to claim 1, wherein the liquid binder is chosen from water, water-miscible organic solvents, oils, and mixtures thereof.

11. The composition according to claim 10, wherein the water-miscible organic solvents are chosen from monoalcohols having from 2 to 6 carbon atoms; polyols having from 2 to 20 carbon atoms; glycol ethers having from 3 to 16 carbon atoms; and mixtures thereof.

12. The composition according to claim 10, wherein the water-miscible organic solvents are chosen from ethanol, isopropanol, glycerol, propylene glycol, butylene glycol, pentylene glycol, hexylene glycol, dipropylene glycol, diethylene glycol, mono-, di- and tripropylene glycol (C₁-C₄)alkyl ethers, mono-, di- and triethylene glycol (C₁-C₄)alkyl ethers, and mixtures thereof.

13. The composition according to claim 10, wherein the oils are chosen from hydrocarbon oils, silicone oils, fluoro oils, and mixtures thereof.

14. The composition according to claim 10, wherein the oils are chosen from volatile oils.

15. The composition according to claim 1, wherein the liquid binder is present in an amount ranging from 5% to 60% by weight, relative to the total weight of the composition.

16. The composition according to claim 1, wherein the colorant comprises reflective particles other than those comprising mica substrates.

17. The composition according to claim 1, wherein the reflective particles other than those comprising mica substrates are chosen from:
particles chosen from natural and synthetic substrates coated at least partially with at least one layer of at least one metal,
particles comprising a synthetic substrate, other than mica, coated at least partially with at least one layer of at least one metal compound,
particles formed from a stack of at least two layers of materials having different refractive indices, wherein at least one layer of the stack of the at least two layers is a polymer;
metal particles; and
mixtures thereof.

18. The composition according to claim 17, wherein the reflective particles are chosen from particles comprising a substrate coated at least partially with at least one layer of at least one metal.

19. The composition according to claim 18, wherein the at least one metal is chosen from Ag, Au, Cu, Al, Zn, Ni, Mo, Cr, and alloys thereof.

20. The composition according to claim 17, wherein the reflective particles comprise a synthetic substrate other than mica, coated at least partially with at least one layer of at least one metal compound.

21. The composition according to claim 20, wherein the at least one metal compound is chosen from titanium oxides, iron oxides, tin oxides, chromium oxides, barium sulphates, compounds MgF₂, CeF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MOS₂, and mixtures thereof.

22. The composition according to claim 21, wherein the at least one metal compound is chosen from titanium oxide, iron oxides, tin oxides, and mixtures thereof.

23. The composition according to claim 20, wherein the synthetic substrate is chosen from substrates comprising a single material or multiple materials, organic substrates, inorganic substrates, glasses, ceramics, metal oxides, aluminas, silicas, and silicates.

24. The composition according to claim 23, wherein the synthetic substrate is chosen from glasses.

25. The composition according to claim 17, wherein the reflective particles are chosen from particles formed by a stack of at least two layers of polymers having different refractive indices.

26. The composition according to claim 17, wherein the reflective particles are chosen from reflective metal particles.

27. The composition according to claim 26, wherein the reflective metal particles are chosen from particles of gold, silver, aluminum, and copper.

28. The composition according to claim 1, wherein the colorant comprises goniochromatic coloring agents.

29. The composition according to claim 28, wherein the goniochromatic coloring agents are pigments having a multi-layer interference structure.

30. The composition according to claim 29, wherein the multi-layer interference structure comprises at least two layers, each layer being made of at least one material chosen from groups of MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cr, Cu, Rb, Ti, Ta, W, Zn, MoS₂, cryolites, alloys, polymers, and combinations thereof.

31. The composition according to claims 29, wherein the multi-layer interference structure is chosen from Al/SiO₂/Al/SiO₂/Al; Cr/MgF₂/Al/MgF₂/Cr; MOS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica-oxide/SiO₂/MOS₂; Fe₂O₃/SiO₂/mica oxide/SiO₂/Fe₂O₃; and combinations thereof.

32. The composition according to claim 28, wherein the goniochromatic coloring agents are liquid-crystal coloring agents.

33. The composition according to claim 32, wherein the liquid-crystal coloring agents are obtained by polymerizing a mixture of monomers comprising:
a) at least one first monomer A of formula (I)
Y1-A1-M1-A2-Y2
wherein
i) Y1 and Y2, which are identical or different, are chosen from acrylate and methacrylate groups;
ii) A1 and A2, which are identical or different, are chosen from groups of formula —CₙH₂ₙ—, wherein n is an integer ranging from 1 to 20;
iii) M1 is a group of general formula (I′)
—R₁—X₁—R₂—X₂—R₃—X₃—R₄—
wherein R₁ and R₄ are —O— atoms, R₂ and R₃ are —COO— groups and X₁, X₂ and X₃ are 1,4-phenylene groups, wherein the carbonyl groups —CO— respectively of R₂ and of R₃ are bonded respectively to the X₁ group and to the X₃ group, and
b) at least one chiral second monomer B of formula (II)
V1 -W1 -Z-W2-V2,
wherein
i) V1 is chosen from acrylate and methacrylate groups, and V2 is chosen from C₁-C₂₀ alkyl groups, C₁-C₂₀ alkoxy groups, (C₁-C₂₀)alkoxy-carbonyl groups, and —OH groups;
ii) W1 is a divalent group of formula —X′1-CO—O—, W2 is a divalent group of formula —O—CO—X′1-, wherein formulae X′1 is a 1,4-phenylene group, and Z is a chiral group comprising two bonds, originating from the dianhydrohexitol group.

34. The composition according to claim 33, wherein the liquid-crystal coloring agents are obtained from a mixture of monomers comprising from 70% to 99% by weight of monomer A and from 1% to 30% by weight of monomer B, relative to the total weight of monomer A and of monomer B.

35. The composition according to claim 1, wherein the colorant comprises photochromic coloring agents.

36. The composition according to claim 35, wherein the photochromic coloring agents are chosen from spiro-oxazines and derivatives thereof; spironaphthoxazines; naphthopyran and derivatives thereof; spiropyrans; nitrobenzylpyridines; spirolanes; titanium oxides and zinc oxides doped with iron; and aluminosilicates doped with metal ions and with a group chosen from Se, S, OH, SO₄²⁻ and WO₄²⁻.

37. The composition according to claim 1, wherein the colorant comprises at least one fluorescent agent.

38. The composition according to claim 37, wherein the at least one fluorescent agent is chosen from organic fluorescent pigments.

39. The composition according to claim 38, wherein the organic fluorescent pigments are chosen from colored polyamide resins, formaldehyde/benzoguanamine resins, melamine/formaldehyde/sulphonamide resins, colored aminotriazine/formaldehyde/sulphonamide co-condensates, metallized polyester flakes, and mixtures thereof.

40. The composition according to claim 37, wherein the at least one fluorescent agent is chosen from optical brighteners.

41. The composition according to claim 40, wherein the optical brighteners are chosen from stilbene derivatives, coumarin derivatives, oxazoles, benzoxazoles, imidazole and triazole derivatives, pyrazolines, pyrene derivatives, porphyrin derivatives, and mixtures thereof.

42. The composition according to claim 40, wherein the optical brighteners are chosen from:
stilbene derivatives of naphtho-triazole, disodium 4,4′-distyrylbiphenylsulphonate, sodium 4,4′-bis[(4,6-dianilino-1,3,5-triazin-2-yl)amino]stilbene-2,2′-disulphonate, 4,4′-bis-[anilino-6-bis(2-hydroxyethyl)amino-1,3,5-triazin-2-yl)amino]stilbene-2,2′-disulphonic acid, 4,4′-bis[(4-anilino-6-morpholine-1,3,5-triazin-2-yl)amino]-stilbene, disodium 4,4′-bis-[(4-anilino-6-hydroxyethyl)methyl amino- 1,3,5-triazin-2-yl)amino]stilbene-2,2′-sulphonate,
2,5-thiophene di-yl bis(5-ter-butyl-1,3-benzoxazole),
anionic derivatives of diaminostilbene dispersed in water, and
mixtures thereof.

43. The composition according to claim 40, wherein the optical brighteners are chosen from disodium 4,4′-distyrylbiphenylsulphonate, sodium 4,4′-bis[4,6-diamilino-1,3,5-triazin-2-yl)amino]stilbene-2,2′-disulphonate, 2,5 thiophene di-yl bis(5 ter-butyl-1,3 benzoxazole), and mixtures thereof.

44. The composition according to claim 1, wherein the colorant is present in an amount ranging from 1% to 80% by weight, relative to the total weight of the composition.

45. The composition according to claim 1, further comprising an additional pulverulent colorant other than the colorants chosen from: reflective particles other than those comprising mica substrates, goniochromatic coloring agents, photochromic coloring agents, fluorescent agents, and mixtures thereof.

46. The composition according to claim 45, wherein the additional pulverulent colorant is chosen from pigments and nacres.

47. The composition according to claim 46, wherein the pigments are chosen from titanium dioxides, zirconium oxides, cerium oxides, zinc oxides, iron oxides, chromium oxides, manganese violet, ultramarine blue, chromium hydrate, and Prussian blue.

48. The composition according to claim 46, wherein the pigments are present in an amount ranging from 0.5% to 30% by weight, relative to the weight of the composition.

49. The composition according to claim 46, wherein the nacres are chosen from mica covered with titanium, mica covered with bismuth oxychlorides, titanium mica covered with iron oxides, titanium mica covered with Prussian blue, titanium mica covered with chromium oxides, titanium mica covered with organic pigments, and nacreous pigments based on bismuth oxychlorides.

50. The composition according to claim 46, wherein the nacres are present in an amount ranging from 0.5% to 50% by weight, relative to the total weight of the composition.

51. The composition according to claim 1, further comprising an additional filler other than elastomeric crosslinked organopolysiloxane particles having a refractive index ranging from 1.2 to 1.45.

52. The composition according to claim 51, wherein the additional filler is chosen from mica, boron nitride, silica particles having a refractive index of more than 1.45, polyamide powders, polyethylene powders, ethylene glycol dimethacrylate-lauryl methacrylate copolymer powders, polymethyl methacrylate particles, and mixtures thereof.

53. The composition according to claim 51, wherein the additional filler is present in an amount ranging from 0.5% to 50% by weight, relative to the total weight of the composition.

54. The composition according to claim 1, wherein a total amount of additional particles other than the elastomeric crosslinked organopolysiloxane particles, ranges from 20% to 80% by weight, relative to the total weight of the composition.

55. The composition according to claim 54, wherein the elastomeric crosslinked organopolysiloxane particles and the additional particles are present in amounts such that the weight ratio of the elastomeric crosslinked organopolysiloxane particles/the colorant and when present, the additional filler and/or the additional pulverulent colorant, ranges from 0.4/1 to 2.5/1.

56. The composition according to claim 1, further comprising at least one cosmetic ingredient chosen from antioxidants, perfumes, preservatives, fibers, neutralizing agents, surfactants, waxes, sunscreens, vitamins, moisturizers, self-tanning compounds, and anti-wrinkle actives.

57. The composition according to claim 1, wherein the composition is in a form chosen from a loose powder, a compact powder, a pressed powder, and a pasty composition.

58. The composition according to claim 1, wherein the composition is a solid composition.

59. The composition according to claim 1, wherein the composition is in a form chosen from a foundation, an eye shadow, a blusher, a concealer product, a body makeup product, and a lip makeup product.

60. A method of making up the skin or lips comprising
applying to the skin or lips a composition comprising
elastomeric crosslinked organopolysiloxane particles having a refractive index ranging from 1.2 to 1.45;
a liquid binder having a refractive index ranging from 1.2 to 1.6; and
a colorant chosen from reflective particles other than those comprising mica substrates, goniochromatic coloring agents, photochromic coloring agents, fluorescent agents, and mixtures thereof.
